(19) United States
(12) Patent Application Publication
(10) Pub. No.: US 2003/0003583 A1
(43) Pub. Date: Jan. 2, 2003

(54) Regulation of transgene expression following AAV transduction

(76) Inventors: Raphael Hirsch, Pittsburg, PA (US); Kristi Jennings, Southgate, KY (US)

Correspondence Address: FROST BROWN TODD LLC, 2200 PNC Center, 201 E. Fifth Street, Cincinnati, OH, 45202-4182, US

(21) Appl. No.: 10/166,536

(22) Filed: Jun. 10, 2002

Related U.S. Application Data
(60) Provisional application No. 60/297,125, filed on Jun. 8, 2001.

(51) Int. Cl. A61K 48/00; C12N 15/861; C12Q 1/68; C12N 9/99

(52) U.S. Cl. 435/456; 424/93.2; 435/184; 435/6

(57) ABSTRACT

The present invention provides for methods for regulating gene expression both in vitro and in vivo. Specifically, the present invention provides for methods of using adeno-associated virus for transduction of a target gene in a variety of tissues wherein the expression of the transgene is regulated by administration of a proteasome inhibitor. As an example, a therapeutic gene can be delivered in vivo by an adeno-associated virus to a tissue that is not normally transduced by adeno-associated virus. The host would then be administered a proteasome inhibitor in order to induce expression of the therapeutic gene. Hence, the proteasome inhibitor would be administered only when gene expression is desired.

### CROSS-REFERENCE TO RELATED APPLICATION

This application claims the benefit of U.S. Provisional Patent Application Serial No. 60/297,125 filed Jun. 8, 2001, which application is hereby incorporated by reference in its entirety.

This invention was made in part with Government support under Grant No. AI40301, awarded by the National Institutes of Health. The Government may have certain rights in this invention.

### FIELD OF THE INVENTION

The present invention provides for methods and compositions for regulating gene expression both in vitro and in vivo. Specifically, the present invention provides for methods of using adeno-associated virus for transduction of a target gene in a variety of tissues wherein the expression of the transgene is regulated by administration of a proteasome inhibitor.

### BACKGROUND OF THE INVENTION

The transfer of genes by means of viruses as a vector is referred to as transduction. Transduction is frequently used to integrate genes into the genome of cells. Adeno-associated viruses (AAV) have been used to construct viral vectors useful for gene therapy. AAV infect various mammalian cells with relatively high efficiency and, in the appropriate host and in the absence of helper virus, can integrate into the host cell genome.

AAVs are single-stranded DNA viruses belonging to the Parvorirus family. In the absence of helper viruses AAVs integrate into the host cell genome. Recombinant AAVs, i.e., AAVs containing foreign DNA, often do not express the foreign DNA in various tissues. However, the latter is important and largely indispensable for gene therapy.

The present invention now provides methods and compositions by which a foreign DNA can be used for gene therapy, under expression regulated conditions, in an effective manner.

### BREIF SUMMARY OF THE INVENTION

The present invention provides for compositions and methods for using adeno-associated virus for transduction of a target gene in a variety of tissues wherein the expression of the transgene is regulated by administration of a proteasome inhibitor.

In one embodiment, a therapeutic gene can be delivered in vivo by an adeno-associated virus to a tissue that is not normally transduced by adeno-associated virus. The host would then be administered a proteasome inhibitor in order to induce expression of the therapeutic gene. Hence, the proteasome inhibitor would be administered only when gene expression is desired.

Accordingly, the present invention provides for methods of using recombinant AAV (rAAV) virions for efficient delivery of genes and sustained production of therapeutic proteins in various cell when regulated using proteasome inhibitors. The invention allows for the secretion of the therapeutic protein from transduced cells such that systemic delivery is achieved. These methods provide for both in vivo and in vitro modes of DNA delivery. Hence, rAAV virions allow delivery of DNA directly to cells, organs and tissues types wherein the ability to express genes in cells, as well as to provide for secretion of the produced protein from such transduced cells, is regulated using proteasome inhibitors and allows the use of gene therapy approaches to treat and/or prevent a wide variety of disorders in a regulated manner.

Thus, in one embodiment, the invention relates to a method of delivering a selected gene of interest to a cell or tissue and selectively regulating its expression. The method comprises: (a) providing a recombinant AAV virion which comprises an AAV vector, the AAV vector comprising the selected gene; (b) introducing the recombinant AAV virion into the cell or tissue; and (c) contacting the cell or tissue with a proteasome inhibitor, wherein the proteasome inhibitor selectively regulates the expression of the selected gene.

In another embodiment, the AAV vector comprises the gene operably linked to control elements capable of directing the in vivo transcription and translation of the selected gene.

Preferably, the proteasome inhibitor is selected from the group consisting of peptidyl aldehydes, boronic acids, boronic esters, lactacystins, β-lactones, vinyl sulfones, peptide boronates and derivatives and analogs thereof. In a preferred embodiment of the invention the peptide aldehyde is an aldehydic tripeptide.

In another embodiment, the invention relates to a method of administering recombinant adeno-associated virus (AAV) virions into the tissue of a mammalian subject, the method comprising: (a) providing AAV virions comprising a selected gene capable of transcription and translation in a desired host cell in vivo; and (b) delivering said recombinant AAV virions to the tissue, (c) administering to the subject a proteasome inhibitor, wherein the proteasome inhibitor regulates the expression of the selected gene and wherein the gene is thereby expressed at a level which provides a therapeutic effect in the mammalian subject. Preferably, the tissue is connective tissue. More preferably, the tissue is synovium.

In one preferred embodiment, the invention relates to a method of selectively regulating the expression of a gene of interest in a transgenic mammal comprising administering a proteasome inhibitor to a transgenic mammal, wherein the proteasome inhibitor regulates the expression of the gene of interest. In another embodiment, the gene of interest is substantially not expressed until contacted with the proteasome inhibitor, which increases the expression of the gene.

In one preferred embodiment, the invention relates to a method of providing recombinant adeno-associated virus (AAV) virions free of both wild-type AAV and infectious helper virus. Preferably, the virions do not generate viral particles. In another embodiment, the recombinant AAV virions are delivered in vivo. In another embodiment, the expression of the transgene is driven by a tissue-specific promoter, which is contained in the vector.

The transgene may be homologous or heterologous to the promoter and/or to the mammal. Typically, the transgene will be a nucleic acid molecule encoding a polypeptide involved in the immune response, hematopoiesis, inflammation, cell growth and proliferation, cell lineage differentiation, and/or the stress response. In one embodiment, the selected gene encodes a therapeutic protein useful for treating a connective tissue disorder.

In particularly preferred embodiments, the selected gene encodes a therapeutic protein, such as erythropoietin, interleukin 1, interleukin 2, interleukin 3, interleukin 4, interleukin 5, interleukin 6, interleukin 7, interleukin 8, interleukin 9, interleukin 10, interleukin 11, interleukin 12, ENA-78, interferon-α, interferon-β, interferon-γ, granulocyte-colony stimulating factor, granulocyte-macrophage colony stimulatory factor, macrophage colony stimulating factor, stem cell factor, keratinocyte growth factor, afamin, monocyte chemoattractant protein, tumor necrosis factor, or biologically active fragments or derivatives of these polypeptides.

In one preferred embodiment, the invention relates to a method of decreasing inflammation in the connective tissue of a patient, comprising delivering a transgene encoding a therapeutic protein useful for treating a connective tissue disorder to at least one connective tissue of the patient, whereby the transgene is delivered to the cells of the connective tissue, and contacting the transgene with a proteasome inhibitor, which increases the expression of the gene, and inflammation in the connective tissue is decreased.

Preferably, the transgene is delivered by recombinant adeno-associated virus (AAV) virions. More preferably, the adeno-associated virus is a replication-deficient adenovirus vector. Generally, about 10³ to about 10¹⁸ recombinant adeno-associated virus vector particles are delivered in vivo. Typically, about 10⁵ to about 10¹⁶ recombinant adeno-associated virus vector particles are delivered in vivo. Preferably, about 10⁷ to about 10¹³ recombinant adeno-associated virus vector particles are delivered in vivo. More preferably, about 10⁹ to about 10¹² recombinant adeno-associated virus vector particles are delivered in vivo.

In one embodiment, vector is predominantly localized to the synovium. In another embodiment, patient has a connective tissue disorder. In yet another embodiment, patient has arthritis.

In still further embodiments, the invention is directed to a method of treating an acquired or inherited disease in a mammalian subject comprising introducing into a cell or tissue of the subject, in vivo, a therapeutically effective amount of a pharmaceutical composition which comprises (a) a pharmaceutically acceptable excipient; and (b) recombinant AAV virions and subsequently contacting the cell or tissue of the subject, in vivo, with a therapeutically effective amount of a pharmaceutical composition which comprises (a) a pharmaceutically acceptable excipient; and (b) a proteasome inhibitor. The recombinant AAV virions comprise an AAV vector, the AAV vector comprising a selected gene capable of transcription and translation of the selected gene when present in the subject. In another embodiment, the recombinant AAV virions comprise an AAV vector, the AAV vector comprising a selected gene operably linked to control elements capable of directing the transcription and translation of the selected gene when present in the subject.

In yet another embodiment, the invention is directed to a method of treating an acquired or inherited disease in a mammalian subject comprising: (a) introducing a recombinant AAV virion into a cell or tissue in vitro to produce a transduced cell. The recombinant AAV virion comprises an AAV vector, the AAV vector comprising a selected gene capable of transcription and translation in the transduced cell when present in the subject; (b) administering to the subject a therapeutically effective amount of a composition comprising a pharmaceutically acceptable excipient and the transduced cells from step (a); and (c) administering to the subject a therapeutically effective amount of a proteasome inhibitor, whereby the selected gene is expressed at a level which provides a therapeutic effect in the mammalian subject.

In a further embodiment, the invention relates to a method for delivering a therapeutically effective amount of a protein systemically to a mammalian subject comprising introducing into a cell or tissue of the subject a pharmaceutical composition which comprises (a) a pharmaceutically acceptable excipient; and (b) recombinant AAV virions, wherein the recombinant AAV virions comprise an AAV vector, the AAV vector comprising a selected gene capable of transcription and translation of the selected gene when present in the subject, wherein the introducing is done in vivo.

In another embodiment, the invention is directed to a method for delivering a therapeutically effective amount of a protein systemically to a mammalian subject comprising: (a) introducing a recombinant AAV virion into a cell or tissue in vitro to produce a transduced cell, wherein the recombinant AAV virion comprises an AAV vector, the AAV vector comprising a selected gene capable of the transcription and translation of the selected gene when present in the subject; (b) administering to the subject a therapeutically effective amount of a composition comprising a pharmaceutically acceptable excipient and the transduced cells from step (a); and (c) administering to the subject a therapeutically effective amount of a proteasome inhibitor, whereby the selected gene is expressed at a level which provides a therapeutic effect in the mammalian subject.

In other embodiments, the invention is directed to an AAV vector comprising a gene encoding a human cytokine operably linked to control elements capable of directing the in vivo transcription and translation of the gene, as well as a recombinant AAV (rAAV) virion comprising the vector.

These and other embodiments of the subject invention will readily occur to those of ordinary skill in the art in view of the disclosure herein.

### BREIF DESCRIPTION OF THE FIGURES

FIG. 1 shows the expression of mouse IL-10 in human type B synoviocytes infected with rAAV(mIL-10) as enhanced by the proteasome inhibitor, zLLL. Synoviocytes were infected with rAAV on day 0. On day 1, cells were exposed to the indicated amounts of zLLL for 24 hours. Supernatants were harvested on days 3 and 7 and assayed by ELISA for mouse IL-10.

FIG. 2 shows the transient and regulatable transgene expression, by zLLL, in human type B synoviocyte cultures infected with rAAV(mIL-10). Synoviocytes were infected with rAAV on day 0. At various times (indicted by the arrows), some cells were exposed to 40 μM of zLLL for 24 hours (A) or to recombinant adenovirus lacking a transgene (B). Supernatants were harvested and assayed by ELISA for mouse IL-10.

FIG. 3 shows the luciferase expression in RA synovium. The in vivo transduction of RA synovium by rAAV is enhanced by the addition of adenovirus particles. RA synovium was implanted subcutaneously into SCID mice. Two weeks later, implants were injected with 10¹⁰ particles of rAAV(luc), encoding the luciferase reporter gene, with or without the addition of 10⁸ particles of the adenovirus Ad(Bg1II). The tissues were removed 2 weeks later and analyzed for luciferase expression.

FIG. 4 shows zLLL upregulates transgene mRNA. Human type B synoviocyte cultures were infected with rAAV(mIL-4). Ten days later, some cells were exposed to 40 μM of zLLL for 24 hours. Cells were harvested and RNA was extracted, reverse transcribed, and subjected to real-time PCR using a LightCycler™ System (Roche Molecular Biochemicals, Indianapolis, Ind.) with the FastStart DNA Master SYBR Green I reaction mix and gene specific primers. LIGHTCYCLER Quantification Software v5.32 was used to compare amplification in the samples during the log-linear phase. Data are normalized to the AAV(mIL-4) samples, which are given a value of 1. The ZLLL group was not infected with AAV (IL-4).

FIG. 5 shows i n vivo administration of zLLL enhances rAAV-mediated transgene expression. RA synovium was implanted into SCID mice. Two weeks later, synovial tissues were injected with 6.8×10¹⁰ particles of AAV(Luc). Two weeks following rAAV administration, zLLL was administered in a 100 μl volume either directly into the synovial tissue or intraperitoneally. Synovial tissues were removed 48 hours later, homogenized, and assayed for luciferase content.

### DETAILED DESCRIPTION OF THE INVENTION

The practice of the present invention will employ, unless otherwise indicated, conventional methods of virology, microbiology, molecular biology and recombinant DNA techniques within the skill of the art. Such techniques are explained fully in the literature.

All publications, patents and patent applications cited herein, whether supra or infra, are hereby incorporated by reference in their entirety. As used in this specification and the appended claims, the singular forms “a,” “an” and “the” include plural references unless the content clearly dictates otherwise.

A. Definitions.

In describing the present invention, the following terms will be employed, and are intended to be defined as indicated below.

By “AAV vector” is meant a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes (described below), but retain functional flanking ITR sequences (also described below). Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

By “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion and both strands are equally infectious.

A used herein, the term “connective tissue” includes but is not limited to a ligament, a cartilage, a tendon, a synovium, skin, bone, meniscus and intervertebral disc tissue of a mammalian host.

As used herein, the term “cytokine” refers to all small proteins with the properties of locally acting hormones. They serve to communicate between cells in a paracrine manner, and may also act in an autocrine manner on the same cell that produces the cytokine(s). Growth factors are types of cytokines that are anti-arthritic in that they maintain synthesis of the cartilaginous matrix. Growth factors include, but are not limited to, transforming growth factor (TGF), TGF-β, TGF-β2 and TGF-β3, fibroblast growth factor (FGF), αFGF and βFGF, insulin-like growth factor (IGF) IGF-1 and IGF-2. Growth hormone, and at least some of the bone morphogenetic proteins (BMP) are also cytokines.

The phrase “delivering a gene” or “transferring a gene” refers to methods or systems for reliably inserting foreign DNA into host cells, such as into cells. Such methods can result in transient or long-term expression of nonintegrated transferred DNA, extrachromosomal replication and expression of transferred replicons (e.g., episodes), or integration of transferred genetic material into the genomic DNA of recipients. Gene transfer provides a unique approach for the treatment of acquired and inherited diseases. A number of systems have been developed for gene transfer into mammalian cells. See, e.g., U.S. Pat. No. 5,399,346.

By “DNA” is meant a polymeric form of deoxyribonucleotides (adenine, guanine, thymine, or cytosine) in double-stranded or single-stranded form, either relaxed and supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence homologous to the MRNA). The term captures molecules that include the four bases adenine, guanine, thymine, or cytosine, as well as molecules that include base analogues which are known in the art.

“Expression control element”, or simply “control element”, refers to DNA sequences, such as initiation signals, enhancers, promoters and silencers, which induce or control transcription of DNA sequences with which they are operably linked. Control elements of a gene may be located in introns, exons, coding regions, and 3′ flanking sequences. Some control elements are “tissue specific”, i.e., and affect expression of the selected DNA sequence preferentially in specific cells (e.g., cells, of a specific tissue), while others are active in many or most cell types. Gene expression occurs preferentially in a specific cell if expression in this cell type is observably higher than expression in other cell types.

A “gene” or “coding sequence” or a sequence which “encodes” a particular protein, is a nucleic acid molecule which is transcribed (in the case of DNA) and translated (in the case of mRNA) into a polypeptide in vitro or In vivo when placed under the control of appropriate regulatory sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

“Homology” refers to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when at least about 80%, preferably at least about 90%, and most preferably at least about 95% of the nucleotides or amino acids match over a defined length of the molecules, as determined using the methods above.

“Initiator” refers to a short, weakly conserved element that encompasses the transcription start site and which is important for directing the synthesis of properly initiated transcripts.

By “mammalian subject” is meant any member of the class Mammalia including, without limitation, humans and non-human primates such as chimpanzees and other apes and monkey species; farm animals such as cattle, sheep, pigs, goats and horses; domestic mammals such as dogs and cats; laboratory animals including rodents such as mice, rate and guinea pigs, and the like. The term does not denote a particular age or sex. Thus, adult and newborn subjects, as well as fetuses, whether male or female, are intended to be covered.

The term “operably linked” refers to the arrangement of various nucleic acid molecule elements relative to each such that the elements are functionally connected and are able to interact with each other. Such elements may include, without limitation, a promoter, an enhancer, a polyadenylation sequence, one or more introns and/or exons, and a coding sequence of a gene of interest to be expressed (i.e., the transgene). The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements.

The term “promoter” refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the transgene.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence.

“Proteasome inhibitor” shall mean any substance which directly or indirectly inhibits the proteasome or the activity thereof. As used herein, proteasome is defined as a multicatalytic proteolytic organelle that is made of multiple subunits of a protein. The proteasome appears to be a major site of degradation for cytosolic proteins.

The term “recombinant AAV virion,” or “rAAV” as used herein encompasses any recombinant AAV gene therapy vehicle of the prior art. More specifically, rAAV defines a recombinant adeno-associated virus comprising: (a) the DNA of, or corresponding to, at least a portion of the genome of an AAV which portion is capable of transducing into a target cell at least one selected gene in the absence of cell division; and (b) at least one selected gene (or transgene) operatively linked to regulatory sequences directing its expression, the gene flanked by the DNA of (a) and capable of expression in the target cell in vivo or in vitro. The construction of recombinant AAV (rAAV) virions has been described. See, e.g., U.S. Pat. Nos. 5,173,414 and 5,139,941.

By “recombinant virus” is meant a virus that has been genetically altered, e.g., by the addition or insertion of a heterologous nucleic acid construct into the particle.

For the purpose of describing the relative position of nucleotide sequences in a particular nucleic acid molecule throughout the instant application, such as when a particular nucleotide sequence is described as being situated “upstream,” “downstream,” “3′,” or “5′” relative to another sequence, it is to be understood that it is the position of the sequences in the “sense” or “coding” strand of a DNA molecule that is being referred to as is conventional in the art.

A “target gene” is a nucleic acid of interest, the expression of which is modulated according to the methods of the invention. The target gene can be endogenous or exogenous and can integrate into a cell's genome, or remain episomal. The target gene can encode, for instance, a protein, an antisense RNA or a ribozyme.

As used herein, the term “therapeutic” refers to the ability of a gene, product, protein, peptide, method and the like to alleviate at least one symptoms of a disorder, or the benefit realized from such alleviation. The term “prophylactic” refers to the ability of a gene, product, protein, peptide, method and the like to prevent or at least retard the onset of at least one symptom of a disorder, or the benefit realized from such action. As used herein, the term “enhanced therapeutic benefit” refers to the therapeutic benefit realized when more than one gene of interest is introduced to a host at the same time; the enhanced therapeutic benefit is greater than the therapeutic benefit of each of the genes administered separately. The benefit can be either additive or synergistic.

The term “therapeutic protein” refers to a protein which is defective or missing from the subject in question, thus resulting in a disease state or disorder in the subject, or to a protein which confers a benefit to the subject in question, such as an antiviral, antibacterial or antitumor function. A therapeutic protein can also be one which modifies any one of a wide variety of biological functions, such as endocrine, immunological and metabolic functions. Representative therapeutic proteins are discussed more fully below.

The term “transduction” denotes the delivery of a DNA molecule to a recipient cell either in vivo or in vitro, via a replication-defective viral vector, such as via a recombinant AAV virion.

The term “transfection” is used to refer to the uptake of foreign DNA by a mammalian cell. A cell has been “transfected” when exogenous DNA has been introduced inside the cell membrane. A number of transfection techniques are known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a plasmid vector and other nucleic acid molecules, into suitable host cells. The term refers to both stable and transient uptake of the genetic material.

The term “transgene” refers to a particular nucleic acid sequence encoding a polypeptide or a portion of a polypeptide to be expressed in a cell into which the nucleic acid sequence is inserted. The term “transgene” is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been inserted; (3) a nucleic acid sequence that serves to add additional copies of the same (i.e., homologous) or a similar nucleic acid sequence naturally occurring in the cell into which it has been inserted; or (4) a silent naturally occurring or homologous nucleic acid sequence whose expression is induced in the cell into which it has been inserted. By “mutant form” is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell.

By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

The present invention provides for methods and compositions for regulating gene expression both in vitro and in vivo. Specifically, the present invention provides for methods of using adeno-associated virus for transduction of a target gene in a variety of tissues wherein the transgene not normally expressed and where the expression is regulated by administration of a proteasome inhibitor. The target gene may encode a surface membrane protein, a secreted protein, a cytoplasmic protein or a ribozyme or an antisense sequence.

Also provided are methods and compositions for producing and using such modified cells. The engineered cells of this invention contain at least one first series of genetic constructs encoding the protein(s) of interest. These constructs are recombinant in the sense that the component portions, e.g. encoding a particular domain or expression control sequence, are not found directly linked to one another in nature (i.e., are heterologous).

In one embodiment, the cells further contain a recombinant genetic construct containing a target gene under the transcriptional control of a transcriptional control element (e.g., promoter/enhancer) responsive to a signal. These constructs are recombinant in the sense that the target gene is not naturally under the transcriptional control of the responsive transcriptional control element.

The constructs of this invention may also contain a selectable marker permitting transfection of the constructs into host cells and selection of transfectants containing the construct. Generally, the vector is an adeno-associated virus (“AAV”). Preferably, the vector is a recombinant adeno-associated virus (“rAAV”).

This invention further encompasses cells containing and/or expressing the construct, including procaryotic and eucaryotic cells and in particular, yeast, worm, insect, mouse or other rodent, and other mammalian cells, including human cells, of various types and lineages, whether frozen or in active growth, whether in culture or in a whole organism containing them

For example, in one aspect, this invention provides cells, preferably but not necessarily mammalian, which contain a first DNA construct comprising a target gene under the transcriptional control of a transcriptional control element responsive to a proteasome inhibitor. The cells further contain a target gene under the expression control of a transcriptional control element responsive to the proteasome inhibitor. Following exposure to the selected proteasome inhibitor, the transgene expresses the target protein.

The AAV-based expression vector to be used typically includes the 145 nucleotide AAV inverted terminal repeats (ITRs) flanking a restriction site that can be used for subdloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb.

In one embodiment, genetically engineered cells of this invention can be used for regulated production of a desired protein. In that embodiment the cells, engineered in accordance with this invention to express a desired gene under proteasome inhibitor-induced regulation, are grown in culture by conventional means. Addition of the proteasome inhibitor to the culture medium leads to expression of the desired gene and production of the desired protein. Expression of the gene and production of the protein can then be turned off by adding to the medium an antagonist reagent or removing the inhibitor. Engineered cells of this invention can also be used in vivo, to modify whole organisms, preferably animals, including humans, e.g. such that the cells produce a desired protein or other result within the animal containing such cells. Such uses include gene therapy. Alternatively, the expressed protein(s) can be used extracellularly to bring together proteins that act in concert to initiate a physiological action.

This invention thus provides materials and methods for achieving a biological effect in cells in response to the addition of a proteasome inhibitor. The method involves providing cells engineered in accordance with this invention and exposing the cells to the proteasome inhibitor.

For example, one embodiment of the invention is a method for activating transcription of a target gene in cells. The method involves providing cells containing and capable of expressing at least one DNA construct encoding a target gene. Preferably, a therapeutic gene is delivered in vivo by an adeno-associated virus to a tissue that is not normally transduced by adeno-associated virus. A tissue that is not normally transduced by adeno-associated virus is one that shows low level expresion of a transgene without the addition of an AAV helper construct (where the helper construct includes AAV coding regions capable of being expressed in the host cell to complement AAV helper functions missing from the AAV vector) or helper viruses such as the adenovirus.

The method thus involves exposing the cells to a proteasome inhibitor in an amount effective to result in expression of the target gene. In cases in which the cells are growing in culture, exposing them to the proteasome adding the proteasome inhibitor to the culture medium effects inhibitor. In cases in which the cells are present within a host organism, administering the proteasome inhibitor to the host organism effects exposing them to the proteasome inhibitor.

The present invention provides for the successful transfer of a selected gene to a cell using recombinant AAV virions. The method allows for the direct, in vivo injection of recombinant AAV virions into tissue, e.g., by intramuscular injection, as well as for the in vitro transduction of cells that can subsequently be introduced into a subject for treatment. The invention also provides for secretion of the produced protein in vivo, from transduced cells, such that systemic delivery can be achieved. Differentiated cells and tissue provide a desirable target for gene therapy since they are readily accessible and nondividing. However, the present invention also finds use with nondifferentiated cells that can be transduced in vitro, and subsequently introduced into a subject.

Preparation of DNA Constructs

This invention contemplates expression of one or more transgenes. In one embodiment, the transgene is delivered to a cell or tissue that is normally not transduced by AAV. Preferably, the transgene is delivered to connective tissue. More preferably, the transgene is delivered to the synovium or synovial cells. Where the transgene is expressed primarily in the synovium, the gene product may be secreted into the bloodstream after synthesis. Thus, included within the scope of this invention is any transgene encoding a polypeptide to be circulated in the blood. Typically, the transgene will be a nucleic acid molecule encoding a polypeptide involved in the immune response, hematopoiesis, inflammation, cell growth and proliferation, cell lineage differentiation, and/or the stress response. The transgene may be homologous or heterologous to the promoter and/or to the mammal. In addition, the transgene may be a full-length cDNA or genomic DNA sequence, or any fragment, subunit or mutant thereof that has at least some biological activity. Optionally, the transgene may be a hybrid nucleic acid sequence, i.e., one constructed from homologous and/or heterologous cDNA and/or genomic DNA fragments. The transgene may also optionally be a mutant of one or more naturally occurring cDNA and/or genomic sequences.

The transgene may be isolated and obtained in suitable quantity using one or more methods that are well known in the art. These methods and others useful for isolating a transgene are set forth, for example, in Sambrook et al. (Molecular Cloning: A Laboratory Manual, Cold Spring Harbor Laboratory Press, Cold Spring Harbor, N.Y., 1989) and in Berger and Kimmel (Methods in Enzymology: Guide to Molecular Cloning Techniques, vol. 152, Academic Press, Inc., San Diego, Calif.).

Where the nucleic acid sequence of the transgene is known, the transgene may be synthesized, in whole or in part, using chemical synthesis methods known in the art. These methods include, inter alia, the phosphotriester, phosphoramidite and H-phosphonate methods of nucleic acid synthesis.

Alternatively, the transgene may be obtained by screening an appropriate cDNA or genomic library using one or more nucleic acid probes (oligonucleotides, cDNA or genomic DNA fragments with an acceptable level of homology to the transgene to be cloned, and the like) that will hybridize selectively with the transgene DNA.

Another suitable method for obtaining a transgene is the polymerase chain reaction (PCR). However, successful use of this method requires that enough information about the nucleic acid sequence of the transgene is known so as to design suitable oligonucleotide primers useful for amplification of the appropriate nucleic acid sequence.

Where the method of choice requires the use of oligonucleotide primers or probes (e.g. PCR, cDNA or genomic library screening), the oligonucleotide sequences selected as probes or primers should be of adequate length and sufficiently unambiguous so as to minimize the amount of non-specific binding that will occur during library screening or PCR. The actual sequence of the probes or primers is usually based on conserved or highly homologous sequences or regions from the same or a similar gene from another organism. Optionally, the probes or primers can be degenerate.

This invention contemplates the use of transgene mutant sequences. A mutant transgene is a transgene containing one or more nucleotide substitutions, deletions, and/or insertions as compared to the wild type sequence. The nucleotide substitution, deletion, and/or insertion can give rise to a gene product (i.e., protein) that is different in its amino acid sequence from the wild type amino acid sequence. Preparation of such mutants is well known in the art.

Typically, the nucleic acid sequence is made up of an enhancer sequence operably linked to a promoter and a transgene. The transgene preferably is a nucleotide sequence encoding a polypeptide involved in immune response, hematopoiesis, inflammation, cell growth and proliferation, cell lineage differentiation, or stress response. The promoter is generally ApoA-I, ApoA-II, ApoA-III, ApoA-IV, ApoB-100, ApoC-I, ApoC-II, ApoC-III, ApoE, albumin, alpha feto protein, PEPCK, transthyretin, SV40, CMV or TK.

Preferably, the polypeptide coded for by the transgenes of the present invention is erythropoietin, interleukin 1, interleukin 2, interleukin 3, interleukin 4, interleukin 5, interleukin 6, interleukin 7, interleukin 8, interleukin 9, interleukin 10, interleukin 11, interleukin 12, ENA-78, interferon-α, interferon-β, interferon-γ, granulocyte-colony stimulating factor, granulocyte-macrophage colony stimulatory factor, macrophage colony stimulating factor, stem cell factor, keratinocyte growth factor, afamin, monocyte chemoattractant protein, tumor necrosis factor, or biologically active fragments or derivatives of these polypeptides.

B. Selection of Regulatory Elements

This invention contemplates the use of promoters that are regulated at least in part by an enhancer that results in increased expression of the transgene. The promoter may be homologous (i.e., from the same species as the mammal to be transfected with the transgene) or heterologous (i.e., from a source other than the species of the mammal to be transfected with the transgene). As such, the source of the promoter may be any unicellular prokaryotic or eukaryotic organism, any vertebrate or invertebrate organism, or any plant, provided that the promoter is functional in combination with an enhancer.

The promoter sequences of this invention may be obtained by any of several methods well known in the art. Typically, promoters useful herein will have been previously identified by mapping and/or by restriction endonuclease digestion and can thus be isolated from the proper tissue source using the appropriate restriction endonucleases. In some cases, the promoter may have been sequenced. For those promoters whose DNA sequence is known, the promoter may be synthesized using the methods described above for transgene synthesis.

Where all or only portions of the promoter sequence are known, the promoter may be obtained using PCR and/or by screening a genomic library with suitable oligonucleotide and/or promoter sequence fragments from the same or another species. Where the promoter sequence is not known, a fragment of DNA containing the promoter may be isolated from a larger piece of DNA that may contain, for example, a coding sequence or even another gene or genes. Isolation may be accomplished by restriction endonuclease digestion using one or more carefully selected enzymes to isolate the proper DNA fragment. After digestion, the desired fragment is isolated by agarose gel purification, Qiagen column or other methods known to the skilled artisan. Selection of suitable enzymes to accomplish this purpose will be readily apparent to one of ordinary skill in the art.

In addition to the transgene, the promoter, and the enhancer, the vectors useful in this invention typically contain one or more other elements useful for (1) optimal functioning of the vector in the mammal into which the vector is transfected, and (2) amplification of the vector in bacterial or mammalian host cells. Each of these elements will be positioned appropriately in the vector with respect to each other element so as to maximize their respective activities. Such positioning is well known to the ordinary skilled artisan. The following elements may be optionally included in the vector as appropriate.

For those embodiments of the invention where the transgene is to be secreted, a signal sequence, is frequently present to direct the polypeptide encoded by the transgene out of the cell where it is synthesized. Typically, the signal sequence is positioned in the coding region of the transgene towards or at the 5′ end of the coding region. Many signal sequences have been identified, and any of them that are functional in the transgenic tissue may be used in conjunction with the transgene. Therefore, the signal sequence may be homologous or heterologous to the transgene, and may be homologous or heterologous to the transgenic mammal. Additionally, the signal sequence may be chemically synthesized using methods set forth above. However, for purposes herein, preferred signal sequences are those that occur naturally with the transgene (i.e., are homologous to the transgene).

In some cases, it may be desirable to have a transgene expressed on the surface of a particular intracellular membrane or on the plasma membrane. Naturally occurring membrane proteins contain, as part of the translated polypeptide, a stretch of amino acids that serve to anchor the protein to the membrane. However, for proteins that are not naturally found on the membrane, such a stretch of amino acids may be added to confer this feature. Frequently, the anchor domain will be an internal portion of the protein and thus will be engineered internally into the transgene. However, in other cases, the anchor region may be attached to the 5′ or 3′ end of the transgene. Here, the anchor domain may first be placed into the vector in the appropriate position as a separate component from the transgene. As for the signal sequence, the anchor domain may be from any source and thus may be homologous or heterologous with respect to both the transgene and the transgenic mammal. Alternatively, the anchor domain may be chemically synthesized using methods set forth above.

An origin of replication element is typically a part of prokaryotic expression vectors purchased commercially, and aids in the amplification of the vector in a host cell. If the vector of choice does not contain an origin of replication site, one may be chemically synthesized based on a known sequence, and ligated into the vector.

A transcription termination element is typically located 3′ to the transgene coding sequence and serves to terminate transcription of the transgene. Usually, the transcription termination element is a polyadenylation signal sequence. While the element is easily cloned from a library or even purchased commercially as part of a vector, it can also be readily synthesized using methods for nucleic acid synthesis such as those described above.

In many cases, transcription of the transgene is increased by the presence of one or more introns on the vector. The intron may be naturally occurring within the transgene sequence, especially where the transgene is a fall length or a fragment of a genomic DNA sequence. Where the intron is not naturally occurring within the DNA sequence (as for most cDNAs), the intron(s) may be obtained from another source. The intron may be homologous or heterologous to the transgene and/or to the transgenic mammal. The position of the intron with respect to the promoter and the transgene is important, as the intron must be transcribed to be effective. As such, where the transgene is a cDNA sequence, the preferred position for the intron is 3′ to the transcription start site, and 5′ to the polyA transcription termination sequence. Preferably for cDNA transgenes, the intron will be located on one side or the other (i.e., 5′ or 3′ ) of the transgene sequence such that it does not interrupt the transgene sequence. Any intron from any source, including any viral, prokaryotic and eukaryotic (plant or animal) organisms, may be used to practice this invention, provided that it is compatible with the host cell(s) into which it is inserted. Also included herein are synthetic introns. optionally, more than one intron may be used in the vector.

Selectable marker genes encode proteins necessary for the survival and growth of transfected cells grown in a selective culture medium. Typical selection marker genes encode proteins that (a) confer resistance to antibiotics or other toxins, e.g., ampicillin, tetracycline, or kanomycin for prokaryotic host cells, and neomycin, hygromycin, or methotrexate for mammalian cells; (b) complement auxotrophic deficiencies of the cell; or (c) supply critical nutrients not available from complex media, e.g., the gene encoding D-alanine racemase for cultures of Bacilli.

All of the elements set forth above, as well as others useful in this invention, are well known to the skilled artisan and are described, for example, in Sambrook et al. (Molecular Cloning: A Laboratory Manual, Cold Spring Harbor Laboratory Press, Cold Spring Harbor, N.Y. [1989]) and Berger et al., eds. (Guide to Molecular Cloning Techniques, Academic Press, Inc., San Diego, Calif.).

C. Construction of Vectors

The vectors most useful in practicing this invention are those that are compatible with prokaryotic cell hosts. However, eukaryotic cell hosts, and vectors compatible with these cells, are within the scope of the invention.

In certain cases, some of the various vector elements may be already present in commercially available vectors such as pUC18, pUC19, pBR322, the pGEM vectors (Promega Corp, Madison, Wis.), the PBLUESCRIPT vectors such as pBIISK+/−(Stratagene Corp., La Jolla, Calif.), and the like, all of which are suitable for prokaryotic cell hosts.

However, where one or more of the elements are not already present in the vector to be used, they may be individually obtained and ligated into the vector. Methods used for obtaining each of the elements are well known to the skilled artisan and are comparable to the methods set forth above for obtaining a transgene (i.e., synthesis of the DNA, library screening, and the like).

Vectors used for amplification of the transgene and/or for transfection of the mammalian embryos are constructed using methods well known in the art. Such methods include, for example, the standard techniques of restriction endonuclease digestion, ligation, agarose and acrylamide gel purification of DNA and/or RNA, column chromatography purification of DNA and/or RNA, phenol/chloroform extraction of DNA, DNA sequencing, polymerase chain reaction amplification, and the like.

The final vector used to practice this invention is typically constructed from a starting vector such as a commercially available vector. This vector may or may not contain some of the elements to be included in the completed vector. If none of the desired elements are present in the starting vector, each element may be individually ligated into the vector by cutting the vector with the appropriate restriction endonuclease(s) such that the ends of the element to be ligated in and the ends of the vector are compatible for ligation. In some cases, it may be necessary to “blunt” the ends to be ligated together in order to obtain a satisfactory ligation. Blunting is accomplished by first filling in “sticky ends” using Klenow DNA polymerase or T4 DNA polymerase in the presence of all four nucleotides. This procedure is well known in the art and is described for example in Sambrook et al., supra.

Alternatively, two or more of the elements to be inserted into the vector may first be ligated together (if they are to be positioned adjacent to each other) and then ligated into the vector.

One other method for constructing the vector to conduct all ligations of the various elements simultaneously in one reaction mixture. Here, many nonsense or nonfunctional vectors will be generated due to improper ligation or insertion of the elements, however the functional vector may be identified and selected by restriction endonuclease digestion.

Transfection of the vector into the selected host cell line accomplished using such methods as calcium phosphate, electroporation, microinjection, lipofection or DEAE-dextran method. The method selected will in part be a function of the type of host cell to be transfected. These methods and other suitable methods are well known to the skilled artisan, and are set forth in Sambrook et al., supra. After culturing the cells long enough for the vector to be sufficiently amplified (usually overnight for E. coli cells), the vector (often termed plasmid at this stage) is isolated from the cells and purified. Typically, the cells are lysed and the plasmid is extracted from other cell contents. Methods suitable for plasmid purification include inter alia, the alkaline lysis mini-prep method.

D. In Vitro and In Vivo Delivery of rAAV Virions

Generally, rAAV virions are introduced into a cell using either in vivo or in vitro transduction techniques. If transduced in vitro, the desired recipient cell will be removed from the subject, transduced with rAAV virions and reintroduced into the subject.

For in vivo delivery of the transgenes, any suitable route of administration may be used, including, direct delivery to the target organ, tissue or site, intranasal, intravenous, intramuscular, subcutaneous, intradermal, vaginal, rectal, and oral administration. Routes of administration may be combined within the course of repeated therapy or immunization. Suitable methods for the delivery and introduction of transduced cells into a subject have been described. For example, cells can be transduced in vitro by combining recombinant AAV virions with cells e.g., in appropriate media, and screening for those cells harboring the DNA of interest using conventional techniques such as Southern blots and/or PCR, or by using selectable markers. Transduced cells can then be formulated into pharmaceutical compositions, described more fully below, and the composition introduced into the subject by various techniques, such as by intramuscular, intravenous, subcutaneous and intraperitoneal injection, or by injection using a catheter.

Pharmaceutical compositions will comprise sufficient genetic material to produce a therapeutically effective amount of the protein of interest, i.e., an amount sufficient to reduce or ameliorate symptoms of the disease state in question or an amount sufficient to confer the desired benefit. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Appropriate doses will depend on the mammal being treated (e.g., human or nonhuman primate or other mammal), age and general condition of the subject to be treated, the severity of the condition being treated, the particular therapeutic protein in question, its mode of administration, among other factors. An appropriate effective amount can be readily determined by one of skill in the art. However, generally, a suitable dose may be in the range of 10³ to 10^{18,} preferably about 10⁵ to 10¹⁶particles per dose, for a subject having a weight of about 80 kg. This dose may be formulated in a pharmaceutical composition, as described above (e.g., suspended in about 0.01 mL to about 1 mL of a physiologically compatible carrier) and delivered by any suitable means. The dose may be repeated, as needed or desired, daily, weekly, monthly, or at other selected intervals.

In another embodiment, the viruses of the invention are useful for ex vivo transduction of target cells. Generally, ex vivo therapy involves removal of a population of cells containing the target cells, transduction of the cells in vitro, and then reinfusion of the transduced cells into the human or veterinary patient. One of skill in the art can readily select ex vivo therapy according to the invention, taking into consideration such factors as the type of target cells to be delivered, the molecule to be delivered, the condition being treated, the condition of the patient, and the like.

Generally, when used for ex vivo therapy, the targeted host cells are infected with 10⁵ viral particles to 10¹⁰ viral particles for each 10¹ to 10¹⁰ cells in a population of target cells. However, other suitable ex vivo dosing levels may be readily selected by one of skill in the art.

In one embodiment, the invention provides methods of delivering transgenes via viral vectors, in which re-administration or repeat delivery is performed using a vector of the invention in which the activating group differs from the activating group utilized on the vector of the first administration.

This method preferably includes introducing the gene encoding the product into at least one target cell of the mammalian host for a therapeutic or prophylactic use. One ex vivo method for treating a connective tissue disorder according to the present invention comprises generating a recombinant vector containing one or more DNA sequences encoding one or more genes of interest, or biologically active derivatives or fragments thereof; infecting a pulsation of in vitro cultured target cells with the vector, resulting in a population of transduced target cells; and transplanting the transduced cells to the mammalian host, effecting subsequent expression of the protein or protein fragment within the host. Expression of the protein or protein fragment of interest is useful in reducing at least one deleterious joint pathology or indicia of inflammation normally associated with a connective tissue disorder. Expression of the DNA sequence can also have a protective effect. Any means known to those skilled in the art can be used to introduce the transduced target cells to the target joint space.

Any type of connective tissue cell or non-connective tissue cells, as those terms are described herein, can be used. Preferably, if using connective tissue, synovial cells are used; more preferably, for treating a human patient, the patient's own cells, such as autologous synovial cells, are used. When ligament cells are used, preferably the ligament is the medial collateral ligament (MCL). Use of cells and/or tissue from the patellar tendon and hamstring are also within the scope of the invention. Preferably, if using non-connective tissue, fibroblast cells are used.

For the ex vivo methods, all of the non-connective tissue cells can be injected back into the bone marrow or bloodstream of the host following transduction. Both connective and non-connective tissue cells can be injected into the joint space, or any other area, of the host following transduction. For the in vivo methods, non-connective tissue cells can be targeted in the bone marrow, bloodstream, joint space, or any other area, of the host and connective tissue cells can be targeted to any area of the host, preferably in the joint space.

Use of numerous genes, and biologically active derivatives and fragments thereof, are within the scope of the invention. Any gene capable of maintenance and expression, and encoding a product having a therapeutic and/or prophylactic effect in the treatment of a pathology can be used in the methods of treating a host. These genes and biologically active derivatives and fragments include, but are not limited to, DNA sequences encoding for one or more cytokine. Any other gene having therapeutic properties and DNA capable of maintenance and expression can also be used. These genes can be either commercially obtained through any supplier or can be made by one skilled in the art from cDNA libraries or through the reverse transcriptase polymerase chain reaction (RTPCR) method. As used herein, the term “cytokine” refers to all small proteins with the properties of locally acting hormones. They serve to communicate between cells in a paracrine manner, and may also act in an autocrine manner on the same cell that produces the cytokine(s). Growth factors are types of cytokines that are anti-arthritic in that they maintain synthesis of the cartilaginous matrix. Growth factors include, but are not limited to, transforming growth factor (TGF), TGF-β, TGF-β2 and TGF-β3, fibroblast growth factor (FGF), αFGF and βFGF, insulin-like growth factor (IGF) IGF-1 and IGF-2.

E. Proteasome Inhibitors

The present invention provides a method for regulating transgene expression in a cell by contacting the cell with an effective amount of a proteasome inhibitor that inhibits a proteasome, so as to thereby increase the transcription of the adeno-associated virus target gene.

Preferably, the proteasome inhibitor is selected from the group consisting of peptidyl aldehydes, boronic acids, boronic esters, lactacystins, betalactones, vinyl sulfones, peptide boronates and derivatives and analogs thereof. In a preferred embodiment of the invention the peptide aldehyde is an aldehydic tripeptide.

In one embodiment the aldehydic tripeptide is N-acetyl-leucyl-leucyl-norleucinal. In another embodiment the aldehydic tripeptide is N-acetyl-leucyl-leucyl-methioninal.

In another embodiment the aldehydic tripeptide is Cbz-leucyl-leucyl-norvalinal (MG115). In another embodiment the aldehydic tripeptide is acetyl-leucyl-leucyl-norvalinal (ALLN). In another embodiment the aldehydic tripeptide is Cbz-ile-glu(O-t-Bu)-ala-leucinal (PSI). In a still further embodiment the aldehydic tripeptide is Cbz-leucyl-leucyl-leucinal (zLLL).

In another embodiment of the invention the proteasome inhibitor is N-acetyl-leucinyl-leucinyl-norleucinyl-H (LLnL) lactacystin, Cbz-LLL, LLnL, MG115, MG132, CEP690, CEP1508, CEP1612, CEP1513, CEP1601, or lactacystin. All of these inhibitors are known in the art (see, e.g., Hughes et al., 1996, J. Exp. Med. 183:1569-1576; Rock et al., 1994, Cell 78:761-771; Yang et al., 1996, J. Exp. Med. 183:1545-1552; Harding et al., 1995, J. Immunol. 22:1767-1775; and Fenteany et al., Science 268: 726-731).

Preferably, the proteasome inhibitor is selected from the group consisting of peptidyl aldehydes, boronic acids, boronic esters, lactacystin, and lactacystin analogs (U.S. Pat. Nos. 6,066,730, 5,780,454, 6,083,903, 6,297,217, 4,499,082, 4,537,773, 5,106,948, 5,169,841, 6,297,217, 6,133,308 and WO 96/32105, WO 96/32105, WO 96/13266, WO 95/24914, WO 91/13904, hereby incorporated by reference in their entirety.)

More preferably, the boronic acid compound for use in the present invention is selected from the group consisting of: N-(4-morpholine) carbonyl-β-(1-naphthyl)-L-alanine-L-leucine boronic acid; N-(8-quinoline)sulfonyl-β-(1-naphthyl)-L-alanine-L-alanine-L-leucine boronic acid; N-(2-pyrazine)carbonyl-L-phenylalanine-L-leucine boronic acid, and N-(4-morpholine)carbonyl-[O-(2-pyridylmethyl)]-L-tyrosine-L-leucine boronic acid.

More preferably, the lactacystin analog is selected from lactacystin, clasto-lactacystin β-lactone, 7-ethyl-clasto-lactacystin ,β-lactone and 7-n-propyl-clasto-lactacystin β-lactone are used for the methods of the invention. Most preferably the lactacystin analog is 7-n-propyl-clasto-lactacystin β-lactone.

Additional compounds can readily be identified as proteasome inhibitors by comparing the activity of putative inhibitors with the activity of known proteasome inhibitors.

Where a proteasome inhibitor is used in the invention, the inhibitor typically is contacted with a cell at a concentration of from about 0.1 μM to 500 μM, preferably at a concentration of from about 0.5 μM to 250 μM, and more preferably at a concentration of from about 1.0 μM to 50 μM. Some are more potent inhibitor, and thus can be used at concentrations as low as from about 10 nM to about 2,000 nM, preferably from about 100 nM to about 1,000 nM, and more preferably from about 500 nM to about 800 nM.

The proteasome inhibitors disclosed herein may be administered by any route, including intradermally, subcutaneously, orally, intraarterially or intravenously. Preferably, administration will be by the intravenous route. Preferably parenteral administration may be provided in a bolus or by infusion.

The concentration of a disclosed proteasome inhibitor in a pharmaceutically acceptable mixture will vary depending on several factors, including the dosage of the compound to be administered, the pharmacokinetic characteristics of the compound(s) employed, and the route of administration. Effective amounts of agents would broadly range between about 10 μg and about 50 mg per Kg of body weight of a recipient mammal. The agent may be administered in a single dose or in repeat doses. Treatments may be administered daily or more frequently depending upon a number of factors, including the overall health of a patient, and the formulation and route of administration of the selected compound(s).

The disclosed proteasome inhibitors may be administered at any time before, during, or after the introduction of the virus. The proteasome inhibitor typically is contacted with a cell at a concentration of 1.0 μM to 50 μM. MG132 is a particularly potent inhibitor, and thus can be used at concentrations as low as 100 nNM to 1,000 nM, preferably 500 nM to 800 nM.

The present invention also provides for a pharmaceutical composition comprising an amount of a proteasome inhibitor capable of passing through a cell membrane and which is effective at inducing expression of a mammalian target gene and a pharmaceutically acceptable carrier. As used herein, “pharmaceutically carriers” means any of the standard pharmaceutically acceptable carriers. Examples include, but are not limited to, phosphate buffered saline, physiological saline, emulsions such as oil and water emulsions and water.

This invention further provide kits for producing cells responsive to a proteasome inhibitor of this invention. One kit contains at least one DNA construct a target gene. The kit may contain a quantity of a proteasome inhibitor of this invention capable of activating expression of the target protein molecules encoded by the DNA constructs of the kit, and may contain in addition a quantity of an antagonist. The kit may farther contain a “second series” DNA construct encoding a target gene and/or transcription control element responsive to the inhibitor. The DNA constructs will preferably be associated with one or more selection markers for convenient selection of transfectants, as well as other conventional vector elements useful for replication in prokaryotes, for expression in eukaryotes, and the like. The selection markers may be the same or different for each different DNA construct, permitting the selection of cells which contain each such DNA construct(s).

Alternatively, a DNA construct for introducing a target gene under the control of a responsive transcriptional control element may contain a cloning site in place of a target gene to provide a kit for engineering cells to inducibly express a gene to be provided by the practitioner.

Any of the kits may also contain positive control cells that were stably transformed with constructs of this invention such that they express a reporter gene in response to exposure of the cells to the proteasome inhibitor. Reagents for detecting and/or quantifying the expression of the reporter gene may also be provided.

The system usually will include a one or more series of constructs, which will provide for expression of one or more genes, usually an exogenous gene. By “exogenous gene” is meant a gene which is not otherwise normally expressed by the cell, e.g. because of the nature of the cell, because of a genetic defect of the cell, because the gene is from a different species or is a mutated or synthetic gene, or the like. Such gene can encode a protein, antisense molecule, ribozyme etc., or can be a DNA sequence comprising an expression control sequence linked or to be linked to an endogenous gene with which the expression control sequence is not normally associated. Thus, as mentioned before, the construct can contain an exogenous or recombinant expression control sequence for proteasome inhibitor-induced expression of an endogenous gene.

The target protein can have an intracellular targeting domain comprising a sequence which directs the chimeric protein to the desired compartment, e.g. surface membrane, nucleus, vesicular membrane, or other site, where a desired physiological activity can be initiated by the proteasome inhibitor.

The cells that may be used in the present invention may be procaryotic, but are preferably eucaryotic, including plant, yeast, worm, insect and mammalian. At present it is especially preferred that the cells be mammalian cells, particularly primate, more particularly human, but can be associated with any animal of interest, particularly domesticated animals, such as equine, bovine, murine, ovine, canine, feline, etc. Among these species, various types of cells can be involved, such as synovium, hematopoietic, neural, mesenchymal, cutaneous, mucosal, stromal, muscle, spleen, reticuloendothelial, epithelial, endothelial, hepatic, kidney, gastrointestinal, pulmonary, etc. Preferably, the cells are fibroblasts. Of particular interest are synoviocyte cells. Also of particular interest are stem and progenitor cells, such as hematopoietic neural, stromal, muscle, hepatic, pulmonary, gastrointestinal, etc. The cells can be autologous cells, syngeneic cells, allogenic cells and even in some cases, xenogeneic cells.

It will generally be necessary to have at least one transcription initiation signal operably linked with the target gene. The expression construct will therefore have at its 5′ end in the direction of transcription, the initiation element and the promoter sequence which allows for induced transcription initiation of a target gene of interest, usually a therapeutic gene. The transcriptional termination region is not as critical, and can be used to enhance the lifetime of or make short half-lived mRNA by inserting AU sequences which serve to reduce the stability of the mRNA and, therefore, limit the period of action of the protein. The initiation or promoter elements can be a single sequence or can be oligomerized, usually having not more than about 5 repeats, usually having about 3 repeats.

A wide variety of genes can be employed as the target gene, including genes that encode a protein of interest or an antisense sequence of interest or a ribozyme of interest The target gene can be any sequence of interest which provides a desired phenotype. The target gene can express a surface membrane protein, a secreted protein, a cytoplasmic protein, or there can be a plurality of target genes, which can express different types of products. The target gene may be an antisense sequence which can modulate a particular pathway by inhibiting a transcriptional regulation protein or turn on a particular pathway by inhibiting the translation of an inhibitor of the pathway. The target gene can encode a ribozyme that may modulate a particular pathway by interfering, at the RNA level, with the expression of a relevant transcriptional regulator or with the expression of an inhibitor of a particular pathway. The proteins which are expressed, singly or in combination, can involve homing, cytotoxicity, proliferation, immune response, inflammatory response, dotting or dissolving of dots, hormonal regulation, or the like. The proteins expressed could be naturally-occurring, mutants of naturally-occurring proteins, unique sequences, or combinations thereof.

The gene can be any gene which is secreted by a cell, so that the encoded product can be made available at will, whenever desired or needed by the host. Various secreted products include hormones, such as insulin, human growth hormone, glucagon, pituitary releasing factor, ACTH melanotropin, relaxin, etc.; growth factors, such as EGF, IGF-1, TGF, PDGF, G-CSF, M-CSF, GM-CSF, FGF, erythropoietin, megakaryocytic stimulating and growth factors, etc.; interleukins, such as IL-1 to -13; TNF, etc.; and enzymes, such as tissue plasminogen activator, members of the complement cascade, perforins, superoxide dismutase, coagulation factors, antithrombin-III, Factor VIIIc, Factor VIIIvW, α-anti-trypsin, protein C, protein S, endorphins, dynorphin, bone morphogenetic protein, CFTR, etc.

The gene can be any gene that is naturally a surface membrane protein or made so by introducing an appropriate signal peptide and transmembrane sequence. Various proteins include homing receptors, e.g. L-selectin (Mel-14), blood-related proteins, particularly having a kringle structure, e.g. Factor VIIIc, Factor VIIIvW, hematopoictic cell markers, e.g. CD3, CD4, CD8, B cell receptor, CD10, CD19, CD28, CD33, CD38, CD41, etc., receptors, such as the interleukin receptors IL-2R, IL4R, etc., channel proteins, for influx or efflux of ions, e.g. H+, Ca+2, K+, Na+, Cl−, etc., and the like; CFTR, activation protein, etc.

Proteins may be modified for transport to a vesicle for exocytosis. By adding the sequence from a protein that is directed to vesicles, where the sequence is modified proximal to one or the other terminus, or situated in an analogous position to the protein source, the modified protein will be directed to the Golgi apparatus for packaging in a vesicle. This process in conjunction with the presence of the chimeric proteins for exocytosis allows for rapid transfer of the proteins to the extracellular medium and a relatively high localized concentration.

Also, intracellular proteins can be of interest, such as proteins in metabolic pathways, regulatory proteins, steroid receptors, transcription factors, etc., particularly depending upon the nature of the host cell. Some of the proteins indicated above can also serve as intracellular proteins.

The constructs can be introduced as one or more DNA molecules or constructs, where there will usually be at least one marker and there may be two or more markers, which will allow for selection of host cells which contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagensis, etc. as appropriate.

The construct(s) once completed and demonstrated to have the appropriate sequences may then be introduced into the host cell by adeno-associated virus (AAV). The host cells will usually be grown and expanded in culture before introduction of the construct(s), followed by the appropriate treatment for introduction of the construct(s) and integration of the construct(s). The cells will then be expanded and screened by virtue of a marker present in the construct. Various markers which may be used successfully include hprt, neomycin resistance, thymidine kinase, hygromycin resistance, etc.

The constructs may be introduced as a single DNA molecule encoding all of the genes, or different DNA molecules having one or more genes. The constructs may be introduced simultaneously or consecutively, each with the same or different markers. Vectors containing useful elements such as bacterial or yeast origins of replication, selectable and/or amplifiable markers, promoter/enhancer elements for expression in procaryotes or eucaryotes, etc. which may be used to prepare stocks of construct DNAs and for carrying out transfections are well known in the art, and many are commercially available.

The cells which have been modified with the DNA constructs are then grown in culture under selective conditions and cells which are selected as having the construct may then be expanded and further analyzed, using, for example, the polymerase chin reaction for determining the presence of the construct in the host cells. Once the modified host cells have been identified, they may then be used as planned, e.g. grown in culture or introduced into a host organism.

The proteasome inhibitor providing for activation of the target gene may then be administered as desired. Depending upon the concentration and type of proteasome inhibitor, the response desired, the manner of administration, the half-life, the number of cells present, various protocols may be employed. The proteasome inhibitor may be administered parenterally or orally. The number of administrations will depend upon the factors described above. The proteasome inhibitor may be taken orally as a pill, powder, or dispersion; bucally; sublingually; injected intravascularly, intraperitoneally, subcutaneously; by inhalation, or the like. The proteasome inhibitors and derivatives may be formulated using conventional methods and materials well known in the art for the various routes of administration. The precise dose and particular method of administration will depend upon the above factors and be determined by the attending physician or human or animal healthcare provider. For the most part, the manner of administration will be determined empirically.

In the event that the activation by the proteasome inhibitor is to be reversed, a compound may be administered that can compete with the proteasome inhibitor. Thus, in the case of an adverse reaction or the desire to terminate the therapeutic effect, an antagonist or inhibiting compound can be administered in any convenient way, particularly intravascularly, if a rapid reversal is desired. Alternatively, one may provide for the presence of an inactivation domain with a DNA binding domain.

The particular dosage of the proteasome inhibitor for any application may be determined in accordance with the procedures used for therapeutic dosage monitoring, where maintenance of a particular level of expression is desired over an extended period of times, for example, greater than about two weeks, or where there is repetitive therapy, with individual or repeated doses of proteasome inhibitor over short periods of time, with extended intervals, for example, two weeks or more. A dose of the proteasome inhibitor within a predetermined range would be given and monitored for response, so as to obtain a time-expression level relationship, as well as observing therapeutic response. Depending on the levels observed during the time period and the therapeutic response, one could provide a larger or smaller dose the next time, following the response. This process would be iteratively repeated until one obtained a dosage within the therapeutic range. Where the proteasome inhibitor is chronically administered, once the maintenance dosage of the proteasome inhibitor is determined, one could then do assays at extended intervals to be assured that the cellular system is providing the appropriate response and level of the expression product.

The subject methodology and compositions may be used for the treatment of a wide variety of conditions and indications, e.g., the treatment of cancer, infectious diseases, metabolic deficiencies, cardiovascular disease, hereditary coagulation deficiencies, autoimmune diseases, joint degenerative diseases, e.g. arthritis, pulmonary disease, kidney disease, endocrine abnormalities, etc. Various cells involved with structure, such as fibroblasts and myoblasts, may be used in the treatment of genetic deficiencies, such as connective tissue deficiencies, arthritis, hepatic disease, etc.

### EXAMPLES

### Example 1

### Expression of Transgene Following AAV-Mediated Gene Transfer in Synovial Cells Exposed to zLLL.

We utilized the proteasome inhibitor, Carbobenzoxyl-L-leucyl-L-leucyl-L-leucinal (zLLL), which has been reported to increase nuclear accumulation of rAAV particles and the rAAV genome in other cell types (Douar, A.

M., K. Poulard, D. Stockholm, and O. Danos. 2001. Intracellular trafficking of adeno-associated virus vectors: routing to the late endosomal compartment and proteasome degradation. J Virol 75:1824-1833; Duan, D., Y. Yue, Z. Yan, J. Yang, and J. F. Engelhardt. 2000. Endosomal processing limits gene transfer to polarized airway epithelia by adeno-associated virus. J Clin Invest 105:1573-1587; Hansen, J., K. Qing, H. J.

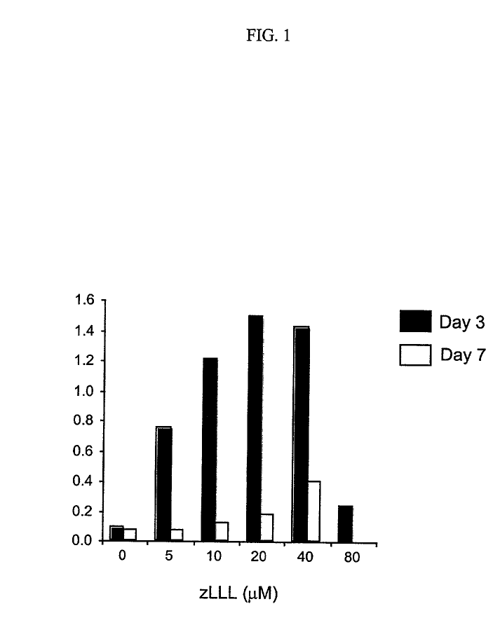

Kwon, C. Mah, and A. Srivastava. 2000. Impaired intracellular trafficking of adeno-associated virus type 2 vectors limits efficient transduction of murine fibroblasts. J Virol 74:992-996) to determine whether human synoviocytes could be made to express a transgene following AAV-mediated gene transfer. As shown in FIG. 1, incubation of human synoviocytes with zLLL dramatically enhanced expression of a mouse IL-10 transgene, 3 days following infection with rAAV, in a dose dependent manner. However, we were surprised to observe that expression was transient, such that by day 7, expression was lost. This is inconsistent with the proposed mechanism of action of zLLL, that of increasing viral trafficking to the nucleus, as transgene expression should be stable once the viral genome reaches the nucleus and sufficient second strand synthesis occurs.

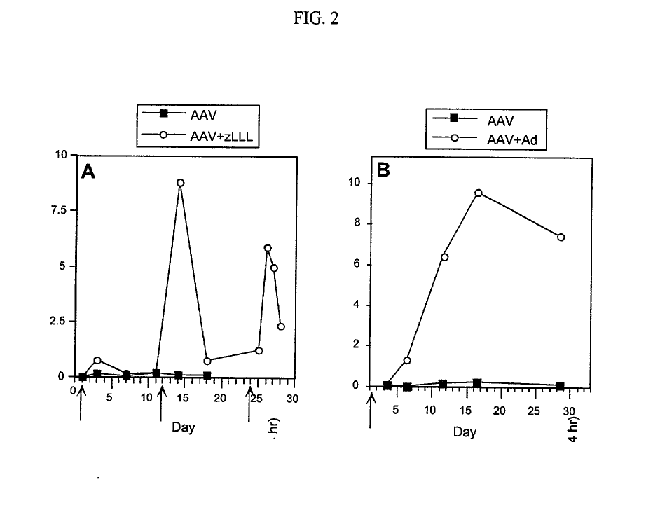

To further explore this finding, synovial cells were re-exposed to zLLL following the loss of transgene expression. We found that transgene expression could be repeatedly induced by re-exposure to zLLL (FIG. 2A). In each case, expression peaked 2-3 days following exposure to zLLL and then dropped off. We were able to re-induce transgene expression as far out as 25 days after infection. We have not yet looked at further time points, but we can already conclude that the transgene persists in the synoviocytes for an extended period of time and that its expression can be regulated by zLLL. This is in contrast to exposure to adenovirus, which results in stable transgene expression (FIG. 2B).

This finding has a number of exciting implications. First, it suggests that zLLL has effects other than enhancement of nuclear transport by which zLLL regulates transgene expression. These could include enhanced transcription, translation, or stability of the transgene product. Potential effects of proteasome inhibitors on protein regulators of gene transcription are of particular interest. For example, they have been shown to influence NFκB-dependent gene transcription by blocking the degradation of IκB (Palombella, V. J., E. M. Conner, J. W. Fuseler, A. Destree, J. M. Davis, F. S. Laroux, R. E. Wolf, J. Huang, S. Brand, P. J. Elliott, D. Lazarus, T. McCormack, L. Parent, R. Stein, J. Adams, and M. B. Grisham. 1998. Role of the proteasome and NF-kappaB in streptococcal cell wall-induced polyarthritis. Proc Natl Acad Sci U S A 95:15671-15676.).

Whatever the mechanism, proteasome inhibitors such as zLLL could be used to regulate transgene expression in human synovium, and possibly other tissues derived from mesenchyme, following rAAV-mediated transduction. zLLL has been successfully used in vivo, both to enhance rAAV-mediated transduction in mouse lungs and similar proteasome inhibitors are being investigated in phase I cancer trials (Adams, J., V. J. Palombella, and P. J. Elliott. 2000. Proteasome inhibition: a new strategy in cancer treatment. Invest New Drugs 18:109-121.). However, no one has suggested that expression can be regulated with these agents. Surprisingly, these agents have little apparent in vivo toxicity (Duan, D., Y. Yue, Z. Yan, J. Yang, and J. F. Engelhardt. 2000. Endosomal processing limits gene transfer to polarized airway epithelia by adeno-associated virus. J Clin Invest 105:1573-1587.).

### Example 2

### Development of a Human-SCID Arthritis Model

In order to study the effects of rAAV on human synovium, an in vivo model would be useful. We have adapted a model in which fragments of human synovial tissue and cartilage are implanted into mice with severe combined immunodeficiency (SCID), which has been successfully used by several groups to study aspects of pathogenesis of RA. Following subcutaneous implantation into SCID mice, RA synovial tissues revascularize and maintain their inflammatory phenotype, including cartilage invasion, for at least 2 months. The vascular and inflamed nature of the synovial implant shows a marked reduction in inflammation following implantation of myocytes expressing vIL-10 (data not shown). Intravenous injection of syngeneic peripheral blood mononuclear cells (PBMC) labeled with fluorescein-5(6)-carboxamido-caproic acid N-hydroxysuccinamide ester (CFSE), which covalently couples to cytoplasmic macromolecules, demonstrates homing to the synovial implants.

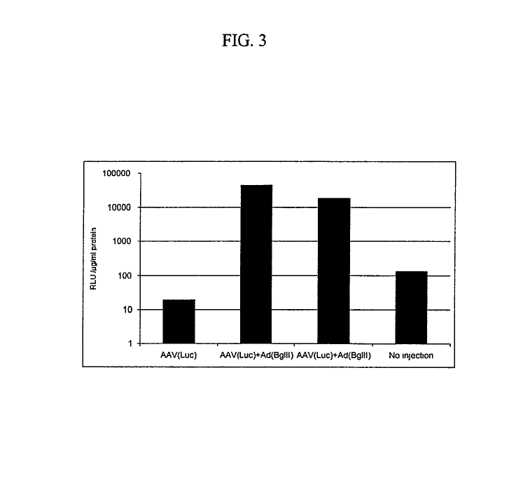

In a preliminary experiment to determine the efficiency of rAAV-mediated transduction of human synovium, RA tissues were implanted into SCID mice. After a 2-week engraftment period, the implants were injected with 10¹⁰ particles of rAAV(luc), encoding the luciferase reporter gene, with or without the addition of 10⁸ particles of Ad(Bg1II). The tissues were removed 2 weeks later and analyzed for luciferase expression. Similar to our in vitro observations, minimal expression was detectable with rAAV alone, however a dramatic increase in expression was observed following co-infection with adenovirus (FIG. 3). A similar finding was observed following injection of rAAV(GFP), encoding green fluorescent protein, or rAAV(LacZ) (data not shown).

### Example 3

### Proteasome Inhibitor Enhancement of rAAV-Mediated Transduction of Human Synoviocytes

The proteasome represents the major nonlysosomal process for degradation of proteins by ATP/ubiquitin-dependent proteolysis. The bulk of proteins in mammalian cells are hydrolyzed by a distinct pathway that requires ATP and the proteasome particle. In this pathway, which is present in both the nucleus and the cytosol, most substrates are first marked for degradation by covalent linkage to multiple molecules of ubiquitin. This includes certain transcription factors and rate-limiting enzymes.

The proteasome inhibitor, Carbobenzoxyl-L-leucyl-L-leucyl-L-leucinal (zLLL), has been reported to increase nuclear accumulation of rAAV particles and the rAAV genome in other cell types. As shown in FIG. 1, incubation of human synoviocytes with zLLL dramatically enhanced expression of a mouse IL-10 transgene, 3 days following infection with rAAV, in a dose dependent manner. However, that expression was transient, such that by day 7, expression was lost. This is inconsistent with the proposed mechanism of action of zLLL, that of increasing viral trafficking to the nucleus, as transgene expression should be stable once the viral genome reaches the nucleus and sufficient second strand synthesis occurs.

To further explore this finding, synovial cells were re-exposed to zLLL following the loss of transgene expression. We found that transgene expression could be repeatedly induced by re-exposure to zLLL (FIG. 2A). In each case, expression peaked 2-3 days following exposure to zLLL and then dropped off. We were able to reinduce transgene expression as far out as 25 days after infection. We have not yet looked at further time points, but we can already conclude that the transgene persists in the synoviocytes for an extended period of time and that its expression can be regulated by zLLL. This is in contrast to exposure to adenovirus, which results in stable transgene expression (FIG. 2B). This effect is not specific for either the IL-10 gene or the CMV promoter, since we observed similar findings using an AAV encoding alkaline phosphatase under control of the rabbit β-globin promoter (data not shown).

zLLL is a peptide aldehyde and can also inhibit certain lysosomal cysteine proteases and the calpains. We have performed a preliminary experiment with Lactacystin, which shows high specificity for the proteasome, with similar results as zLLL (data not shown).

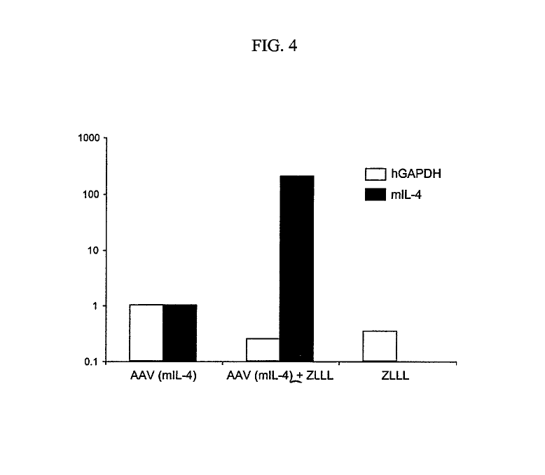

Preliminary data suggests that the zLLL-induced enhancement of transgene product may be mediated by upregulation of gene transcription or mRNA stability. Synoviocytes transduced with AAV(mIL-4) were exposed to zLLL and RNA was extracted and subjected to real-time PCR. zLLL induced a 250-fold increase in mIL-4 mRNA with no increase in the constituitively-expressed gene, GAPDH (FIG. 4). This does not rule out the possibility that the effect is mediated by enhanced transport of the AAV genome to the nucleus, which will require quantitation of viral cytoplasmic and nuclear DNA.

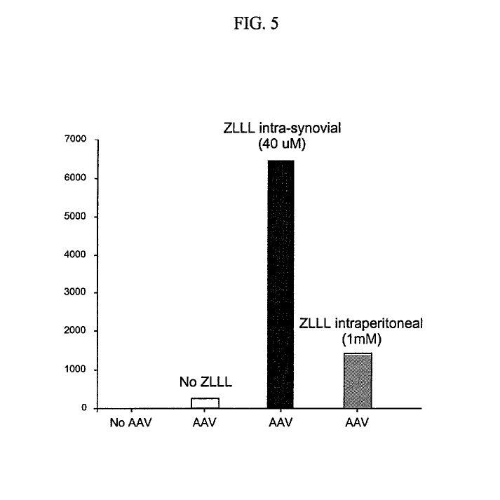

zLLL also dramatically enhanced in vivo transgene expression (FIG. 5). The optimal in vivo dose and route of administration is unclear from this preliminary study and will be addressed in the proposed experiments.

What is claimed is:

1. A method of delivering a selected gene to a cell and modulating its expression, comprising (a) providing a recombinant adeno-associated virus virion which comprises an adeno-associated virus vector, the adeno-associated virus vector comprising the selected gene; (b) introducing the recombinant adeno-associated virus virion into the cell; and (c) contacting the cell with a proteasome inhibitor, wherein the proteasome inhibitor modulates the expression of the selected gene.

2. The method of claim 1, wherein the adeno-associated virus vector comprises the selected gene operably linked to control elements capable of directing the in vivo transcription and translation of the selected gene.

3. The method of claim 2, wherein the proteasome inhibitor is used at a concentration effective to specifically modulate expression of the selected gene and thereby affect the level of the protein encoded by the selected gene that is expressed by the cell.

4. The method of claim 3 wherein the modulation of gene expression is increasing of gene expression.

5. The method of claim 4, wherein the proteasome inhibitor is selected from the group consisting of peptidyl aldehydes, boronic acids, boronic esters, lactacystins, β-lactones, vinyl sulfones, peptide boronates and derivatives and analogs thereof.

6. The method of claim 4, wherein the proteasome inhibitor is a peptidyl aldehyde.

7. The method of claim 4, wherein the proteasome inhibitor is a peptidyl boronic acid or peptidyl boronic ester.

8. The method of claim 4, wherein the proteasome inhibitor is selected from the group consisting of: N-(4-morpholine)carbonyl-β-(1-naphthyl)-L-alanine-L-leucine boronic acid; N-(8-quinoline)sulfonyl-β-(1-naphthyl)-L-alanine-L-alanine-L-leucine boronic acid; N-(2-pyrazine)carbonyl-L-phenylalanine-L-leucine boronic acid, and N-(4-morpholine)carbonyl-[O-(2-pyridylmethyl)]-L-tyrosine-L-leucine boronic acid.

9. The method of claim 4, wherein the proteasome inhibitor is a lactacystin analog.

10. The method of claim 9 wherein the lactacystin analog is selected from lactacystin, clasto-lactacystin β-lactone, 7-ethyl-clasto-lactacystin β-lactone and 7-n-propyl-clasto-lactacystin β-lactone.

11. The method of claim 4, wherein the proteasome inhibitor is an aldehydic tripeptide.

12. The method of claim 11, wherein the aldehydic tripeptide is selected from the group consisting of N-acetyl-leucyl-leucyl-norleucinal, N-acetyl-leucyl-leucyl-methioninal, Cbz-leucyl-leucyl-norvalinal (MG115), acetyl-leucyl-leucyl-norvalinal (ALLN), Cbz-ile-glu(O-t-Bu)-ala-leucinal (PSI), Cbz-leucyl-leucyl-leucinal (zLLL).

13. The method of claim 4, wherein the proteasome inhibitor is lactacystin.

14. The method of claim 5, wherein the cell is contacted with a concentration of proteasome inhibitor of from about 0.01 μM to about 500 μM.

15. The method of claim 5, wherein the cell is contacted with a concentration of proteasome inhibitor of from about 0.5 μM to about 250 μM,

16. The method of claim 5, wherein the cell is contacted with a concentration of proteasome inhibitor of from about 1.0 μM to about 50 μM.

17. The method of claim 5, wherein the cell is a cell of a multicellular organism.

18. The method of claim 5, wherein the cell is a fibroblast.

19. The method of claim 5, wherein the vector comprises an enhancer sequence operably linked to a promoter and a transgene.

20. The method of claim 19, wherein the promoter is selected from the group consisting of ApoA-I, ApoA-II, ApoA-III, ApoA-IV, ApoB-100, ApoC-I, ApoC-II, ApoC-III, ApoE, albumin, alpha feto protein, PEPCK, transthyretin, SV40, CMV and TK.

21. The method of claim 5, wherein the selected gene is a human gene.

22. The method of claim 21, wherein the selected gene is a nucleotide sequence encoding a polypeptide involved in a physiological processes selected from the group consisting of immune response, hematopoiesis, inflammation, cell growth and proliferation, cell lineage differentiation, and stress response.

23. The method of claim 21, wherein the polypeptide coded for by the transgene is selected from the group consisting of erythropoietin, interleukin 1, interleukin 2, interleukin 3, interleukin 4, interleukin 5, interleukin 6, interleukin 7, interleukin 8, interleukin 9, interleukin 10, interleukin 11, interleukin 12, ENA-78, interferon-α, interferon-β, interferon-γ, granulocyte-colony stimulating factor, granulocyte-macrophage colony stimulatory factor, macrophage colony stimulating factor, stem cell factor, keratinocyte growth factor, afamin, monocyte chemoattractant protein, tumor necrosis factor, transforming growth factor (TGF), fibroblast growth factor (FGF), insulin-like growth factor (IGF) and biologically active fragments and derivatives of these polypeptides.

24. The method of claim 21, wherein the polypeptide coded for by the transgene is a secreted product selected from the group consisting of insulin, human growth hormone, glucagon, pituitary releasing factor, ACTH melanotropin, relaxin, etc.; growth factors, such as EGF, IGF-1, TGF, PDGF, G-CSF, M-CSF, GM-CSF, FGF, erythropoietin, megakaryocytic stimulating and growth factors, etc.; interleukins, such as IL-1 to -13; TNF, tissue plasminogen activator, members of the complement cascade, perforins, superoxide dismutase, coagulation factors, antithrombin-III, Factor VIIIc, Factor VIIIvW, α-anti-trypsin, protein C, protein S, endorphins, dynorphin, bone morphogenetic protein, CFTR, and biologically active fragments and derivatives of these polypeptides.

25. The method of claim 21, wherein the polypeptide coded for by the transgene is a surface membrane protein selected from the group consisting of homing receptors,, blood-related proteins, hematopoietic cell markers, cell receptors, channel proteins, for influx or efflux of ions, CFTR, activation protein, and biologically active fragments and derivatives of these polypeptides.

26. A method of administering recombinant adeno-associated virus virions into the tissue of a mammalian subject and modulating its expression, the method comprising: (a) providing adeno-associated virus virions comprising a selected gene capable of transcription and translation in a desired host cell in vivo; and (b) delivering the recombinant adeno-associated virus virions to the tissue, (c) administering to the subject a proteasome inhibitor, wherein the proteasome inhibitor is used at a concentration effective to specifically modulate expression of the selected gene and thereby affect the level of the protein encoded by the selected gene that is expressed by the tissue

27. The method of claim 26, wherein the gene is thereby expressed at a level that provides a therapeutic effect in the mammalian subject.

28. The method of claim 26, wherein the adeno-associated virus vector comprises the selected gene operably linked to control elements capable of directing the in vivo transcription and translation of the selected gene.

29. The method of claim 27, wherein the proteasome inhibitor is used at a concentration effective to specifically modulate expression of the selected gene and thereby affect the level of the protein encoded by the selected gene that is expressed by the tissue.

30. The method of claim 29, wherein the modulation of gene expression is increasing of gene expression.

31. The method of claim 30, wherein the tissue is connective tissue.

32. The method of claim 30, wherein the tissue is synovium.

33. The method of claim 30, wherein the proteasome inhibitor is selected from the group consisting of peptidyl aldehydes, boronic acids, boronic esters, lactacystins, β-lactones, vinyl sulfones, peptide boronates and derivatives and analogs thereof.

34. The method of claim 30, wherein the proteasome inhibitor is selected from the group consisting of: N-(4-morpholine)carbonyl- β-(1-naphthyl)-L-alanine-L-leucine boronic acid; N-(8-quinoline)sulfonyl-β-(1-naphthyl)-L-alanine-L-alanine-L-leucine boronic acid; N-(2-pyrazine)carbonyl-L-phenylalanine-L-leucine boronic acid, and N-(4-morpholine)carbonyl-[O-(2-pyridynaethyl)]-L-tyrosine-L-leucine boronic acid.

35. The method of claim 30, wherein the proteasome inhibitor is a lactacystin analog.

36. The method of claim 35., wherein the lactacystin analog is selected from lactacystin, clasto-lactacystin, β-lactone, 7-ethyl-clasto-lactacystin β-lactone and 7-n-propyl-clasto-lactacystin β-lactone.

37. The method of claim 30, wherein the proteasome inhibitor is an aldehydic tripeptide.

38. The method of claim 37, wherein the aldehydic tripeptide is selected from the group consisting of N-acetyl-leucyl-leucyl-norleucinal, N-acetyl-leucyl-leucyl-methioninal, Cbz-leucyl-leucyl-norvalinal (MG115), acetyl-leucyl-leucyl-norvalinal (ALLN), Cbz-ile-glu(O-t-Bu)-ala-leucinal (PSI), Cbz-leucyl-leucyl-leucinal (zLLL).

39. The method of claim 30, wherein the proteasome inhibitor is administered by the route selected from intrademially, subcutaneously, orally, intraarterially and intravenously.

40. The method of claim 33, wherein the proteasome inhibitor is administered at a dose from about 10 μg to about 50 μg per Kg of body weight of a mammalian subject.

41. The method of claim 33, wherein the selected gene is a human gene.

42. The method of claim 42, wherein the selected gene is a nucleotide sequence encoding a polypeptide involved in a physiological processes selected from the group consisting of immune response, hematopoiesis, inflammation, cell growth and proliferation, cell lineage differentiation, and stress response.

43. The method of claim 41, wherein the polypeptide coded for by the transgene is selected from the group consisting of erythropoietin, interleukin 1, interleukin 2, interleukin 3, interleukin 4, interleukin 5, interleukin 6, interleukin 7, interleukin 8, interleukin 9, interleukin 10, interleukin 11, interleukin 12, ENA-78, interferon-α, interferon-β, interferon-γ, granulocyte-colony stimulating factor, granulocyte-macrophage colony stimulatory factor, macrophage colony stimulating factor, stem cell factor, keratinocyte growth factor, afamin, monocyte chemoattractant protein, tumor necrosis factor, transforming growth factor (TGF), fibroblast growth factor (FGF), insulin-like growth factor (IGF) and biologically active fragments and derivatives of these polypeptides.

44. The method of claim 41, wherein the polypeptide coded for by the transgene is a secreted product selected from the group consisting of insulin, human growth hormone, glucagon, pituitary releasing factor, ACTH melanotropin, relaxin, etc.; growth factors, such as EGF, IGF-1, TGF, PDGF, G-CSF, M-CSF, GM-CSF, FGF, erythropoietin, megakaryocytic stimulating and growth factors, etc.; interleukins, such as IL-1 to -13; TNF, tissue plasminogen activator, members of the complement cascade, perforins, superoxide dismutase, coagulation factors, antithrombin-III, Factor VIIIc, Factor VIIIvW, α-anti-trypsin, protein C, protein S, endorphins, dynorphin, bone morphogenetic protein, CFTR, and biologically active fragments and derivatives of these polypeptides.

45. A method of selectively regulating the expression of a gene of interest in a transgenic mammal comprising administering a proteasome inhibitor to a transgenic mammal, wherein the proteasome inhibitor regulates the expression of the gene of interest.

46. The method of claim 45, wherein the gene of interest is substantially not expressed until contacted with the proteasome inhibitor, which increases the expression of the gene.

47. The method of claim 45, wherein the gene of interest is provided by recombinant adeno-associated virus virions free of both wild-type adeno-associated virus and infectious helper virus.

48. The method of claim 47, wherein the virions do not generate viral particles.

49. The method of claim 48, wherein the recombinant adeno-associated virus virions are delivered in vivo.

50. The method of claim 49, wherein the expression of the transgene is driven by a tissue-specific promoter, which is contained in the vector.

51. The method of claim 50, wherein the gene is homologous to the mammal.

52. The method of claim 50, wherein the gene is heterologous to the promoter.

53. The method of claim 50, wherein the transgene is a nucleic acid molecule encoding a polypeptide involved in the immune response, hematopoiesis, inflammation, cell growth and proliferation, cell lineage differentiation, and/or the stress response.

54. The method of claim 50, wherein the selected gene encodes a therapeutic protein useful for treating a connective tissue disorder.

55. The method of claim 54, wherein the selected gene encodes a therapeutic protein, such as erythropoietin, interleukin 1, interleukin 2, interleukin 3, interleukin 4, interleukin 5, interleukin 6, interleukin 7, interleukin 8, interleukin 9, interleukin 10, interleukin 11, interleukin 12, ENA-78, interferon-α, interferon-β, interferon-γ, granulocyte-colony stimulating factor, granulocyte-macrophage colony stimulatory factor, macrophage colony stimulating factor, stem cell factor, keratinocyte growth factor, afamin, monocyte chemoattractant protein, tumor necrosis factor, or biologically active fragments or derivatives of these polypeptides.

56. The method of claim 50, wherein the gene is thereby expressed at a level that provides a therapeutic effect in the mammalian subject.

57. The method of claim 50, wherein the adeno-associated virus vector comprises the selected gene operably linked to control elements capable of directing the in vivo transcription and translation of the selected gene.

58. A method of decreasing inflammation in the connective tissue of a patient, comprising delivering a transgene encoding a therapeutic protein useful for treating a connective tissue disorder to at least one connective tissue of the patient, whereby the transgene is delivered to the cells of the connective tissue, and contacting the tissue with a proteasome inhibitor, which increases the expression of the transgene, and inflammation in the connective tissue is decreased.

59. The method of claim 58, wherein the transgene is delivered by recombinant adeno-associated virus virions.

60. The method of claim 59, wherein the adeno-associated virus is a replication-deficient adenovirus vector.

61. The method of claim 60, wherein about 10³ to about 10¹⁸ recombinant adeno-associated virus vector particles are delivered in vivo.

62. The method of claim 60, wherein about 10⁵ to about 10¹⁶ recombinant adeno-associated virus vector particles are delivered in vivo.

63. The method of claim 60, wherein about 10⁷ to about 10¹³ recombinant adeno-associated virus vector particles are delivered in vivo.

64. The method of claim 60, wherein about 10⁹ to about 10¹² recombinant adeno-associated virus vector particles are delivered in vivo.

65. The method of claim 60, wherein the vector is predominantly localized to the synovium.

66. The method of claim 60, wherein the patient has a connective tissue disorder.

67. The method of claim 60, wherein the patient has arthritis.

68. A method of treating an acquired or inherited disease in a mammalian subject comprising introducing into a cell or tissue of the subject, in vivo, a therapeutically effective amount of a pharmaceutical composition which comprises (a) a pharmaceutically acceptable excipient; and (b) recombinant adeno-associated virus virions and subsequently contacting the cell or tissue of the subject, in vivo, with a therapeutically effective amount of a pharmaceutical composition which comprises (a) a pharmaceutically acceptable excipient; and (b) a proteasome inhibitor. The recombinant adeno-associated virus virions comprise an adeno-associated virus vector, the adeno-associated virus vector comprising a selected gene capable of transcription and translation of the selected gene when present in the subject.

69. The method of claim 68, wherein the adeno-associated virus vector comprises a selected gene operably linked to control elements capable of directing the transcription and translation of the selected gene when present in the subject.

70. A method of treating an acquired or inherited disease in a mammalian subject comprising: (a) introducing a recombinant adeno-associated virus virion into a cell or tissue in vitro to produce a transduced cell, the recombinant adeno-associated virus virion comprises an adeno-associated virus vector, the adeno-associated virus vector comprising a selected gene capable of transcription and translation in the transduced cell when present in the subject; (b) administering to the subject a therapeutically effective amount of a composition comprising a pharmaceutically acceptable excipient and the transduced cells from step (a); and (c) administering to the subject a therapeutically effective amount of a proteasome inhibitor, whereby the selected gene is expressed at a level which provides a therapeutic effect in the mammalian subject.

71. The method of claim 70, wherein the recombinant AAV virions are delivered to the synovium cells in vitro and the synovium cells are delivered directly into synovium of the subject.

72. A method for delivering a therapeutically effective amount of a protein systemically to a mammalian subject comprising (a) introducing into a cell or tissue of the subject a pharmaceutical composition which comprises (i) a pharmaceutically acceptable excipient; and (ii) recombinant adeno-associated virus virions, wherein the recombinant adeno-associated virus virions comprise an adeno-associated virus vector, the adeno-associated virus vector comprising a selected gene capable of transcription and translation of the selected gene when present in the subject and wherein the introducing is done in vivo; and (b) administering to the subject a therapeutically effective amount of a proteasome inhibitor, whereby the selected gene is expressed at a level which provides a therapeutic effect in the mammalian subject.

73. The method of claim 72, wherein the recombinant AAV virions are delivered to the synovium cells in vitro and the synovium cells are delivered directly into synovium of the subject.

74. The method of claim 72, wherein the targeted subject cells are infected with 10⁵ viral particles to 10¹⁰ viral particles for each 10¹ to 10¹⁰ cells in a population of subject cells.

75. A method for delivering a therapeutically effective amount of a protein systemically to a mammalian subject comprising: (a) introducing a recombinant adeno-associated virus virion into a cell or tissue in vitro to produce a transduced cell, wherein the recombinant adeno-associated virus virion comprises an adeno-associated virus vector, the adeno-associated virus vector comprising a selected gene capable of the transcription and translation of the selected gene when present in the subject; (b) administering to the subject a therapeutically effective amount of a composition comprising a pharmaceutically acceptable excipient and the transduced cells from step (a); and (c) administering to the subject a therapeutically effective amount of a proteasome inhibitor, whereby the selected gene is expressed at a level which provides a therapeutic effect in the mammalian subject.

76. The method of claim 75, wherein the recombinant AAV virions are delivered to the synovium cells in vitro and the synovium cells are delivered directly into synovium of the subject.

77. The method of claim 75, wherein the targeted subject cells are infected with 10⁵ viral particles to 10¹⁰ viral particles for each 10¹ to 10¹⁰ cells in a population of subject cells.

78. The method of claim 75, wherein the adeno-associated virus vector comprises a gene encoding a human cytokine operably linked to control elements capable of directing the in vivo transcription and translation of the gene, as well as a recombinant adeno-associated virus virion comprising the vector.

79. The method of claim 78, wherein the selected gene encodes a therapeutic protein useful for treating a connective tissue disorder.

80. The method of claim 79, wherein the therapeutic protein is selected from the group consisting of erythropoietin, interleukin 1, interleukin 2, interleukin 3, interleukin 4, interleukin 5, interleukin 6, interleukin 7, interleukin 8, interleukin 9, interleukin 10, interleukin 11, interleukin 12, ENA-78, interferon-α, interferon-β, interferon-γ, granulocyte-colony stimulating factor, granulocyte-macrophage colony stimulatory factor, macrophage colony stimulating factor, stem cell factor, keratinocyte growth factor, afamin, monocyte chemoattractant protein, tumor necrosis factor, transforming growth factor (TGF), fibroblast growth factor (FGF), insulin-like growth factor (IGF) and biologically active fragments and derivatives of these polypeptides.

81. The method of claim 79, wherein the gene encodes a secreted protein.

82. A method of expressing a therapeutically effective amount of a protein in a mammalian subject, the method comprising (a) administering into the bloodstream of the subject a pharmaceutical composition which comprises (i) a pharmaceutically acceptable excipient; and (ii) recombinant adeno-associated virus virions comprising a selected gene operably linked to expression control elements that provide for transcription and translation of the selected gene in a desired host cell in vivo, whereby the virions transduce cells in the subject, and (b) administering to the subject a therapeutically effective amount of a proteasome inhibitor, whereby the selected gene is expressed by the transduced cells at a level which provides for a therapeutic effect in the mammalian subject.

83. The method of claim 82, wherein the pharmaceutical composition is delivered intraarterially.

84. The method of claim 82, wherein the pharmaceutical composition is delivered intravenously.

85. The method of claim 82, wherein the transgene comprises a nucleotide sequence encoding a polypeptide involved in immune response, hematopoiesis, inflammation, cell growth and proliferation, cell lineage differentiation, or stress response.

86. The method of claim 82, wherein the promoter is selected from the group of promoters consisting of the promoters of ApoA-I, ApoA-II, ApoA-III, ApoA-IV, ApoB-100, ApoC-I, ApoC-II, ApoC-III, ApoE, albumin, alpha feto protein, PEPCK, transthyretin, SV40, CMV and TK.

87. The composition of claim 82, wherein the polypeptide of interest is a therapeutic agent.

88. The method of claim 87, wherein the proteasome inhibitor is selected from the group consisting of peptidyl aldehydes, boronic acids, boronic esters, lactacystins, β-lactones, vinyl sulfones, peptide boronates and derivatives and analogs thereof.

89. The method of claim 88, wherein the proteasome inhibitors is administered by the route selected from intradermally, subcutaneously, orally, intraarterially and intravenously.

90. The method of claim 89, wherein the effective amounts of agents would broadly range between about 10 μg and about 50 mg per Kg of body weight of a recipient mammal.

91. The method of claim 1 further comprising the step of administering a second agent.
